(19) United States
(12) Patent Application Publication
(10) Pub. No.: US 2019/0376115 A1
(43) Pub. Date: Dec. 12, 2019

(54) ACTIVITY SENSOR DESIGN

(71) Applicant: GLYMPSE BIO, INC., Cambridge, MA (US)

(72) Inventors: Sangeeta Bhatia, Lexington, MA (US); Gabriel Kwong, Atlanta, GA (US); Eric Huang, Lexington, MA (US); Sirshendu Roopom Banerjee, Lexington, MA (US); Andrew Warren, Cambridge, MA (US); Sophie Cazanave, Cambridge, MA (US)

(21) Appl. No.: 16/434,785

(22) Filed: Jun. 7, 2019

Related U.S. Application Data
(60) Provisional application No. 62/682,507, filed on Jun. 8, 2018.

(51) Int. Cl. C12Q 1/37; C12Q 1/6886; G16B 40/00; G16B 50/00

(52) U.S. Cl. CPC C12Q 1/37; C12Q 1/6886; G01N 2333/948; G16B 50/00; C12Q 2600/158; G16B 40/00

(57) ABSTRACT

Methods of the disclosure provide an analytical pipeline for mapping activity in a disease-specific manner. Any of a variety of diseases or medical conditions may be mapped using the analytical pipeline. In preferred embodiments, the pipeline uses expression data (e.g., from RNA-Seq) to identify proteases that are active in disease tissue and subject to differential expression relative to normal tissue. A machine learning classifier selects a subset of the proteases that identify the disease with a threshold sensitivity and specificity, in which the subset is small enough that a corresponding set of protease substrates may be assembled into a nanoparticle activity sensor that, when administered to a patient, are cleaved in the presence of disease tissue to release detectable analytes signifying presence of the disease.

### CROSS-REFERENCE TO RELATED APPLICATION

This application claims priority to U.S. Provisional Application Ser. No. 62/682,507, filed Jun. 8, 2018, incorporated by reference.

### TECHNICAL FIELD

The disclosure relates to design methodologies for activity sensors that can report a physiological state in a subject with sensitivity and specificity.

### BACKGROUND

Current approaches to detecting or diagnosing diseases such as cancer involve techniques such as obtaining a tissue biopsy and examining cells under a microscope or sequencing DNA to detect genetic markers of the disease. It is thought that early detection is advantageous because some treatments will have a greater chance of success with early intervention. For example, with cancer, a tumor may be surgically removed and a patient may go into full remission if the cancer is detected before it spreads throughout the body in a process known as metastasis. Medical consensus is that outcomes such as remission after tumor resection require early detection.

Unfortunately, existing approaches to disease detection do not always detect a disease at its incipiency. For example, while x-ray mammogram represents an advance over manual examination in that an x-ray may detect a tumor that cannot be detected by physical examination. Such tests nevertheless require a tumor to have progressed to some degree for detection to occur. Liquid biopsy represents one potential method for disease detection. In a liquid biopsy, a blood sample is taken and screened for small fragments of tumor DNA using next-generation sequencing instruments. Liquid biopsy offers the potential for relatively early detection of a tumor as it is understood that a growing tumor will have cells that rupture and release DNA fragments into the bloodstream. As long as a tumor has grown to a sufficient degree, there is a possibility that liquid biopsy could detect its presence. Unfortunately, x-ray mammogram, microscopic examination of tissue samples, and liquid biopsy do not always detect disease as early as would be most medically beneficial.

### SUMMARY

The invention provides methods for designing biological activity sensors that reveal activity inside of the body that is predictive of a physiological state such as a specific disease or stage of a disease. The activity sensors can be provided as small nanosensors that, when administered to a patient, traffic to tissue where they are cleaved by enzymes that are differentially expressed in tissue of the physiological state to release detectable analytes. The detectable analytes are excreted in a bodily sample such as urine, sweat, or breath where they are detected and show the presence of the disease. For any given disease, the activity sensors are designed by a process that includes testing tissue samples to identify enzymes that are expressed under disease conditions. A classification algorithm is used to select a set of those enzymes that are specific to the disease condition, and the activity sensor is created that releases its panel of detectable analytes only in the presence of that set of enzymes.

The design method may be implemented in a bioinformatics pipeline that uses input data such as sequences generated by expression profiling of diseased tissue by RNA-Seq or the results from a proteomics assays, such as the use of DNA-barcoded antibodies. The pipeline can output a set of enzymes specific for a disease or even for a stage of a disease, or the pipeline can output specific design parameters for the activity sensor, such as polypeptide sequences to be included for cleavage by the enzymes. The pipeline can beneficially output a heat map that maps substrate space to protease space, i.e., to indicate what peptides to include in activity sensors to provide activity sensors that report a given physiological state. An axis of a heat map can include proteases that are differentially expressed (e.g., both up-regulated and down-regulated) under a physiological state against an axis for peptide substrates. Moreover, the pipeline can include the classifier algorithm that detects the requisite subset of enzymes that serve as markers of a specific disease or disease stage, and distinguish the condition from healthy tissue, with reproducible sensitivity and specificity.

By providing gene expression information as input to the informatics pipeline, one may reliably identify a short list of enzymes that characterizes tissue as being affected by disease at a given stage. Additionally, the pipeline is a design tool for biological activity sensors in that it determines peptides that will be cleaved from an activity sensor by the specific enzymes to release analytes that can be detected to report the presence of the disease. The pipeline is a tool for creating the activity sensor as, once the determined peptides are known, one may synthesize the peptides and attach them to a biocompatible scaffold to form a nanoparticle for administration to a patient. By including peptides with enzyme-specific cleavage substrates, the activity sensor will release the panel of detectable analytes in the presence of those disease-associated analytes.

By controlling properties of the scaffold and releasable analytes, such as mass and size, an activity sensor can be made that will locate to the specific tissue or tumor and release the detectable analytes. The released analytes may be detected by a suitable assay such as mass spectrometry or an ELISA blot.

The activity sensors give an amplified signal in the presence of the enzymes. Because the activity sensors may include a plurality of substrates for any one enzyme, the presence of even a very small quantity of that enzyme will release an abundance of detectable analyte. The activity sensors are well suited for detection of diseases that advance via the release of extracellular tissue re-modeling enzymes. Such disease include cancer, in which extracellular proteases digest and cleave connective tissue at a very early stage to allow a tumor to grow and penetrate into the tissue. Activity sensors designed according to the disclosure are very sensitive and suited for detection of disease at its earliest stages, long before, for example, a tumor has grown to a point at which it can be detected by other methods.

The activity sensors may be used to stage disease with precision. When the classification algorithm of the design pipeline is applied to data of the heat maps of enzyme activity by disease stage, the pipeline reliably finds a subset of the enzymes that is specific for a disease at a given stage. Thus the design pipeline can be used to create an activity sensor that will show the stage of a cancer of a specific tissue, or show the stage of advancement of other disease such as liver disease, including for example nonalcoholic steatohepatitis (NASH), even a specific stage of NASH. Thus the disclosure provides a rational design methodology for the creation of tools for non-invasive early disease detection, staging, and monitoring. The design methodology may be implemented in an automated analytical pipeline using expression data such as RNA-Seq results or a proteomics assay as inputs to map activity of diseased tissue to create the sensitive and precise activity sensors.

In certain aspects, the invention provides methods for designing activity sensors. Methods include analyzing gene expression of tissue in a disease state to identify enzymes such as proteases that are differentially expressed in the tissue compared to healthy tissue, selecting a subset of the enzymes that correlates with the disease state to a predefined threshold of sensitivity or specificity, and creating an activity sensor comprising cleavable reporters that are released as analytes in vivo upon exposure to the subset of enzymes. When the activity sensor is administered to a patient, proteases cleave the activity sensor in the tissue affected by the disease and release the analyte for collection in a bodily sample.

In some embodiments, the subset of enzymes is selected by a machine learning classification algorithm that classifies subsets by whether they meet the threshold sensitivity or specificity. The classification algorithm may use or create a heat map that gives an expression level of each enzyme at stages of the disease. Preferably, the classification algorithm outputs a set of proteases predicted to classify the disease condition with sensitivity and specificity both greater than 0.90 per an area under a receiver-operating curve (AUROC). The method may include selecting the cleavage targets as substrates for the proteases output by the classification algorithm.

In certain embodiments, analyzing the gene expression includes sequencing RNA from disease tissue samples to produce transcript sequences. A computer system may be used to compare the transcript sequences, or translations thereof, to a gene or protein database to identify candidate proteases. The RNA-Seq may be performed using suitable input samples such as formalin-fixed, paraffin-embedded slices from tumors.

Methods preferably include creating the activity sensor. Where the enzymes are proteases, creating the activity sensor may include linking a plurality of peptides to a polymer scaffold. Each of the peptides may have a detectable analyte linked to the scaffold via a cleavage target of one of the signature proteases. In some embodiments, the polymer scaffold comprises a multi-arm (PEG) structure. Administering the activity sensor to a patient yields a bodily sample from the subject that includes the analytes, indicating disease activity before other disease symptoms are exhibited by the subject.

In certain embodiments, the bioinformatics pipeline is trained and developed using tissue data in which the disease is nonalcoholic steatohepatitis (NASH). The differentially expressed enzymes (i.e., differentially expressed in diseased versus normal tissue) include FAP, MMP2, ADAMTS2, FURIN, MMP14, GZMB, PRSS8, MMP8, ADAM12, CTSS, CTSA, CTSZ, CASP1, ADAMTS12, CTSD, CTSW, MMP11, MMP12, GZMA, MMP23B, MMP7, ST14, MMP9, MMP15, ADAMDEC1, ADAMTS1, GZMK, KLK11, MMP19, PAPPA, CTSE, PCSK5, and PLAU, and the machine learning classifier identified the classifying subset of enzymes as several or all of FAP, MMP2, ADAMTS2, FURIN, MMP14, MMP8, MMP11, CTSD, CTSA, MMP12, and MMP9. In other embodiments, the disease is lung cancer, and the classifying subset of enzymes may include, for example, MMP13, MMP11, MMP12, MMP1, KLK6, and MMP3.

Preferably, the pipeline is used to design activity sensor that report a plurality of differentially expressed proteases in which different ones of the proteases are included for distinct informatics content. For example, certain of the proteases can be up-regulated in a certain disease, while certain ones may be down-regulated and, additionally, other ones of the proteases may be differentially expressed under certain stages of certain tissue conditions. Additionally, one or more proteases may be probed for that are not differentially expressed under the physiological condition and whose activity thus provides a baseline to be subtracted out of the others, or for normalizing the others.

Any suitable disease may be profiled including, for example, cancer, osteoarthritis, or pathogen infection. In staging embodiments, the enzymes are proteases and the method includes determining subsets of the proteases specific to disease stages, wherein administering the activity sensor to a subject yields a bodily sample with analytes indicative of a stage of the disease.

Aspects of the disclosure provide a system for designing an activity sensor. The system includes at least one computer comprising a processor coupled to memory having instructions therein executable by the processor to cause the system to analyze gene expression of tissue in a disease state to identify enzymes differentially expressed in the tissue compared to healthy tissue and select a subset of the enzymes that correlates with the disease state to threshold sensitivity or specificity. The system stores or outputs a set of enzymes specific for a disease or even for a stage of a disease, or specific design parameters for the activity sensor, such as polypeptide sequences to be included for cleavage by the enzymes. The system may include instruments such as nucleic acid sequencing instruments to perform RNA-Seq to determine the gene expression levels from the tissue. wherein analyzing the gene expression includes sequencing RNA from disease tissue samples to produce transcript sequences. The system may use the transcript sequences, or translations thereof, to query a gene or protein database to identify candidate proteases. The system may provide outputs to laboratory instruments used for creating an activity sensor comprising cleavable reporters that are released as analytes in vivo upon exposure to the subset of enzymes. The system selects the subset of enzymes using a machine learning classification algorithm that classifies subsets by whether they meet the threshold sensitivity or specificity. The system may provide a heat map that gives an expression level of each enzyme at stages of the disease. Preferably, the classification algorithm outputs a set of proteases predicted to classify the disease condition with sensitivity and specificity both greater than 0.90 (and actually achieved better than 0.93), wherein each of the peptides comprises a detectable analyte linked to the scaffold via a cleavage target of one of the signature proteases. In some embodiments, the system automatically determines and outputs the cleavage targets, i.e., the sequences for substrates for the proteases output by the classification algorithm.

In an exemplary embodiment, the system provides an informatics pipeline used to analyze expression data from tissue samples affected by a target disease of interest. From the expression data (e.g., RNA-Seq data), the system identifies all proteases expressed in disease-affected tissue, i.e., by look-up to a database or list. A differential expression module in the pipeline outputs a list with e.g., tens, dozens, or more enzymes that are expressed differentially in disease versus healthy tissue. A classifier module such as a trained machine learning algorithm selects a set of enzymes (e.g., between about 5 and about 20, preferably about 8 to 12) that, when detected in tissue, reliably report the presence or specific stage of the disease to a threshold sensitivity and specificity demonstrable by an AUROC better than 0.90. The system may be used to determine targets for cancer, osteoarthritis, or pathogen infection.

In certain aspects, the invention provides a method for designing activity sensors based on collateral cleavage. The method includes analyzing gene expression data for tissue affected by a disease condition to identify candidate genes differentially expressed in the tissue compared to healthy tissue, identifying a set of signature genes that classify the disease condition with a threshold sensitivity or specificity, and creating a composition that, when administered to the subject, releases one or more detectable reporters in the presence of nucleic acid sequences of the signature genes. The composition may include a Cas protein that exhibits collateral cleavage in the presence of the nucleic acid sequences of the signature genes. In some embodiments, the composition includes reporters that include quenched fluorophores that fluoresce in response to collateral cleavage by the Cas protein. In certain embodiments, the composition includes a plurality of the Cas proteins, and the composition provides a fluorescent signature that classifies the disease based on exposure of the Cas proteins to sequences of the signature genes.

### BRIEF DESCRIPTION OF THE DRAWINGS

FIG. 1 diagrams a method for designing an activity sensor.

FIG. 2 shows analyzing gene expression of tissue samples.

FIG. 3 shows an exemplary list of proteases.

FIG. 4 is a graph of classification accuracy.

FIG. 5 shows the results of the classification algorithm.

FIG. 6 shows an activity sensor.

FIG. 7 shows an 8-arm PEG scaffold.

FIG. 8 illustrates an exemplary mass spectra obtained from a patient.

FIG. 9 illustrates a system according to certain embodiments.

FIG. 10 shows an activity sensor created by the method used to detect disease.

FIG. 11 shows proteases that exhibit differential expression.

FIG. 12 shows the results of staging NASH using activity sensors.

FIG. 13 shows results from validating the activity sensors in mice.

FIG. 14 shows the 156 differentially expressed extracellular proteases.

FIG. 15 shows upregulated genes.

FIG. 16 shows an activity map, or heat map, generated from analysis of RNA-Seq data.

### DETAILED DESCRIPTION

Methods of the disclosure provide an analytical pipeline for mapping activity in a disease-specific manner. Any of a variety of diseases or medical conditions may be mapped using the analytical pipeline. In preferred embodiments, the pipeline uses expression data (e.g., from RNA-Seq or a proteomics assay) to identify proteases that are active in disease tissue and subject to differential expression relative to normal tissue. A machine learning classifier selects a subset of the proteases that identify the disease with a threshold sensitivity and specificity, in which the subset is small enough that a corresponding set of protease substrates may be assembled into a nanoparticle activity sensor that, when administered to a patient, are cleaved in the disease tissue to release detectable analytes signifying presence of the disease. A pipeline generally refers to a series of analytical steps or data processing elements (modules, code blocks, programs) connected in series, generally on a computer hardware platform such as a server which may be a dedicated server or a cloud server that adds virtual machines on demand. In an informatics pipeline, a sequence of computing processes (commands, program runs, tasks, threads, procedures, etc.) are executed in parallel and or series to identify sets of protease substrates. In the pipeline, the output stream of one process is preferably automatically fed as the input stream of the next one such that, for example, RNA-Seq reads are passed to an assembler or mapper, which passes transcript sequences to a database look-up module that identifies a full set of proteases. That module passes the proteases to the machine learning classifier which converges on a set of, e.g., 10 or 12 proteases that identify a disease or stage to the threshold sensitivity or specificity. The informatics pipeline may further include a database lookup (i.e., to query online databases) or an internal look-up table in a module that give protease substrates (peptide sequence data) as outputs when given protease names as inputs.

Any suitable tools or development environment may be used to implement the pipeline. For example, for some embodiments, a pipeline was developed in the R computing environment and implemented using a library of packages such as the open source software package Bioconductor. Bioconductor provides tools for the analysis and comprehension of high-throughput genomic data. Bioconductor uses the R statistical programming language, and is open source and open development. It has two releases each year, 1560 software packages, and an active user community. Bioconductor is also available as an AMI (Amazon Machine Image) and a series of Docker images. See Huber, 2015, Orchestrating high-throughput genomic analysis with Bioconductor, Nat Meth 12:115-121 and Gentleman, 2004, Bioconductor: open software development for computational biology and bioinformatics, Genome Biology 5:r80, both incorporated by reference. In particular, the pipeline used the Bioconductor packages DE-seq and caret (for classification). The pipeline is preferably optimized for highly expressed and highly differential expression transcripts. A pipeline of the disclosure may be implemented on a server and may automatically receive data such as RNA-Seq inputs and use packages and wrapper scripts to process the data to produce outputs for the design of nanosensors/activity sensors.

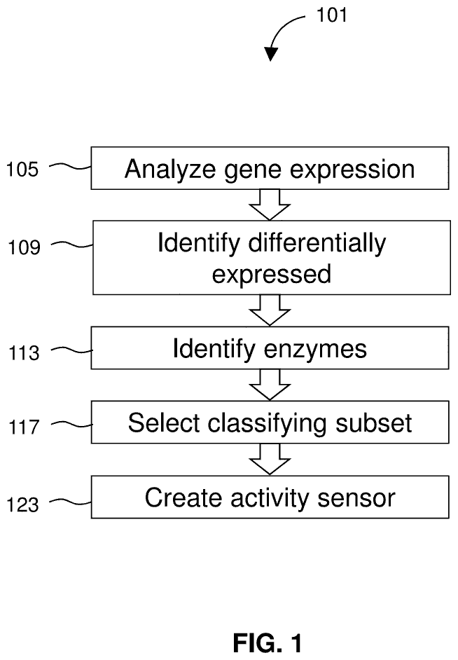

FIG. 1 diagrams a method 101 for designing an activity sensor. The method 101 includes analyzing 105 gene expression of tissue in a disease state to identify 113 enzymes, and to identify 109 those enzymes that are differentially expressed in the tissue compared to healthy tissue. The method 101 further includes selecting 117 a subset of the enzymes that correlates with the disease state to threshold sensitivity or specificity and creating 123 an activity sensor comprising cleavable reporters that are released as analytes in vivo upon exposure to the subset of enzymes. The steps are preferably performed in an informatics pipeline on a computer server and begin with obtaining gene expression for tissue of known disease status.

Any suitable technique for analyzing 105 gene expression from disease affected tissue may be used. For example, gene expression data may be obtained from a database of results, or diseased tissue may be analyzed for proteins present, e.g., by a hybridization assay or by a mass spectrometry assay. In certain embodiments, gene expression is analyzed by a proteomic assay of a sample to identify proteins or enzymes that are present. In certain embodiments, a proteomics assay uses fluorescently-labelled and/or DNA-barcoded antibodies to detect proteins. For example, the proteins may be detected using the materials, methods, and instruments for proteomics assays sold under the trademark NANOSTRING by NanoString Technologies, Inc. (Seattle, Wash.). See WO 2007/076129 A2; U.S. 2010/0015607 A1; U.S. 2010/0047924 A1; WO 2010/019826 A1; WO 2011/116088 A2; U.S. 2011/0229888 A1; WO 2012/178046 A2; U.S. 2013/0017971 A1; and U.S. Pat. No. 8,519,115 B2, all incorporated by reference. Gene expression data may be obtained via fluorescent in-situ hybridization. In some embodiments, gene expression is analyzed by RNA-Seq from tissue sample.

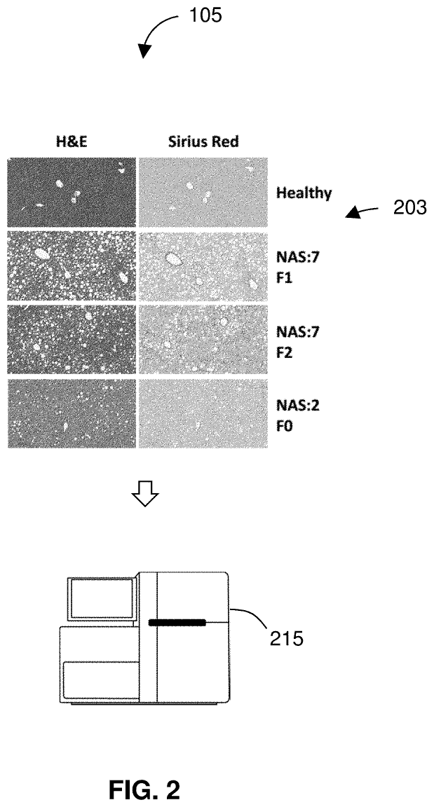

FIG. 2 shows analyzing 105 gene expression of tissue samples 203 in a disease state to identify enzymes differentially expressed in the tissue compared to healthy tissue. RNA sequencing (RNA-Seq) may be performed on RNA extracted from procured formalin fixed and paraffin embedded (FFPE) liver tissue from patients with the disease of interest. RNA may be isolated from tissue and mixed with deoxy-ribonuclease to isolate the RNA. To analyze signals of interest, the isolated RNA can either be kept as is, filtered for RNA with 3′ polyadenylated (poly(A)) tails to include only mRNA, depleted of ribosomal RNA (rRNA), and/or filtered for RNA that binds specific sequences. The RNA with 3′ poly(A) tails are mature, processed, coding sequences. Poly(A) selection is performed by mixing RNA with poly(T) oligomers covalently attached to a substrate, typically magnetic beads. Poly(A) selection ignores noncoding RNA and is followed by cDNA synthesis. The RNA is reverse transcribed to cDNA. Fragmentation and size selection may be performed to purify sequences that are the appropriate length for the sequencing instrument 215. The RNA, cDNA, or both are fragmented with enzymes, sonication, or nebulizers. The cDNA for each experiment can be indexed with a hexamer or octamer barcode, so that these experiments can be pooled into a single lane for multiplexed sequencing. See Wang, 2009, RNA-Seq: a revolutionary tool for transcriptomics, Nat Rev Genet 10(1):57-63, incorporated by reference.

Sequencing produces a number of sequence reads. The sequence reads may be assembled to reconstruct sequences of the transcripts that were present in the tissue samples 203. Assembling sequence reads may be performed by a computer system of the invention using known assembly methods including de novo assembly by a multiple sequence alignment, mapping to a reference genome, assembly suing internal barcodes, or combinations thereof. Sequence assembly may use any methods such as those described in U.S. Pat. No. 8,209,130, incorporated by reference. Analyzing the gene expression of the tissue samples 203 preferably provides transcript sequences. Methods may include comparing the transcript sequences, or translations thereof, to a gene or protein database to identify candidate proteases. Using NASH as an example, a plurality of proteases may be identified.

In certain embodiments, RNA Seq data is assembled into transcript sequences. Those may be, for example, FASTA files. In one embodiment, a query module performs BLAST for each transcript against a source such as GenBank and retrieves gene names and identifies proteases. In a preferred embodiment, the informatics pipeline includes a file of sequences and names of the approximately 200 extracellular proteases that have been identifies, sequenced, and annotated. A module compares the transcript sequences to the file in a pairwise fashion using BLAST or a similar alignment-based comparison algorithm (e.g., Smith-Waterman) and returns the names of those proteases that were identified as present in the disease tissue. The pipeline compares the results (e.g., expression levels from RNA-Seq) from disease tissue to those from healthy tissue and outputs a list of proteases differentially expressed in disease versus healthy tissue.

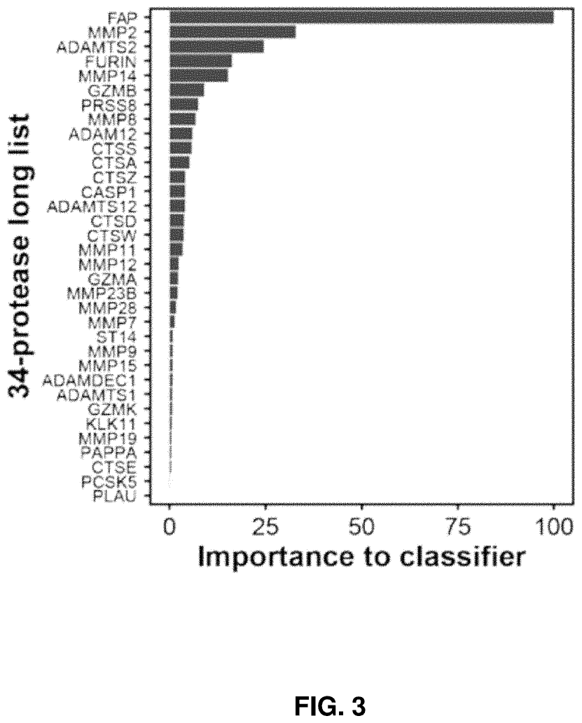

FIG. 3 shows an exemplary list of proteases that may be identified as differentially expressed in a disease condition (NASH) compared to in health tissue. The proteases are used in designing an activity sensor. One insight of the disclosure is that an activity sensor may give good results by including a certain number of protease substrates. For any given protease, if the activity sensor includes the cleavage substrate of that protease in a number of duplicates, the protease will catalyze cleavage of all or many of the duplicate substrates. Even if a single molecule of protease is present, substantially all of the substrate may be cleaved, and a concomitant quantity detectable analyte may be released. If the activity sensor is delivered at such does that, on average, 10,000 copies of the sensor come into proximity with the site of protease expression, and if each sensor has, on average 1 copy of the substrate, that dosage should yield substantially on the order of 10,000 copies of detectable analyte. If each activity sensor has a geometry and chemistry to support linkage to between 1 and 20 unique cleavage substrates/detectable analytes, then it may be desirable to select about, for example, ten or so unique protease substrates for attachment to the sensors.

The disclosure further includes the discovery that such numbers of proteases (e.g., about 8, or about 10, or about 12, 15, 18, etc.) statistically give precise and sensitive signatures of disease as shown by AUROCs better than 0.9. Accordingly, where the differential expression analysis reports 30 or 50 or more proteins (e.g., see the 34 proteases differentially expressed in NASH shown in FIG. 3) that are differentially expressed under a disease condition, a classification algorithm may be applied to identify a subset that operates as a disease signature, wherein the subset includes a number of proteases the activity of which uniquely and reliably identifies a given disease at a given stage.

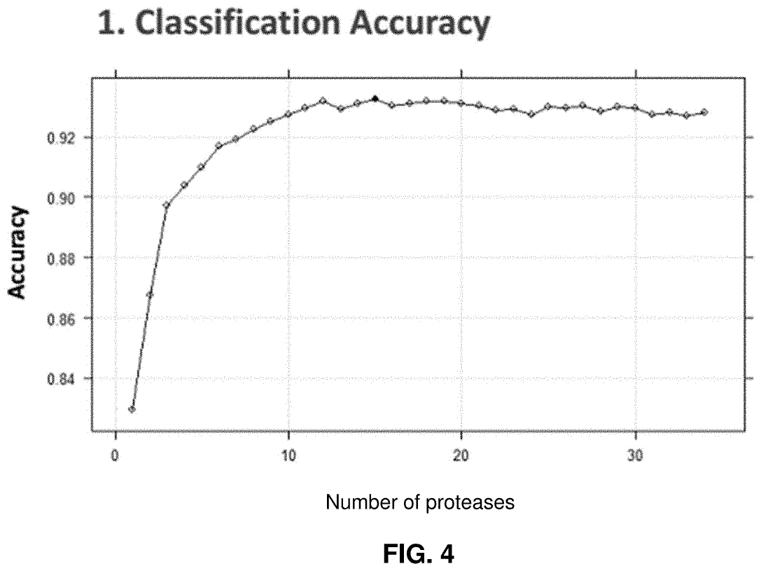

FIG. 4 is a graph of classification accuracy over number of proteases for NASH. The disclosure includes the discovery that classification accuracy stabilizes as a number N of proteases approaches 10. It may be found that a preferred number of proteases to probe for via an activity sensor is about 10, e.g., 8, 9, 10, 11, or 12. A computer system of the disclosure may be used for selecting a subset of the enzymes that correlates with the disease state to threshold sensitivity or specificity. In preferred embodiments, the subset of enzymes is selected by a machine learning classification algorithm that classifies subsets by whether they meet the threshold sensitivity or specificity. For example, a machine learning algorithm can use the transcript sequences from the RNA-Seq data from tissue and healthy samples as training data. The algorithm can sample subsets of the proteases and determine correlations to the known disease status (disease or disease stage versus healthy).

Any suitable machine learning classifier may be used to select sets of proteases. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. For example, a neural network may be used to select protease sets.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, L. Random Forests, Machine Learning 45:5-32 (2001), incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

A support vector machine (SVM) may be used to classify subsets of proteases as predictive of disease or disease state. A SVM creates a hyperplane in multidimensional space that separates data points into one category or the other. Although the original problem may be expressed in terms that require only finite dimensional space, multidimensional space may be selected to allow construction of hyperplanes that afford clean separation of data points. SVMs can also be used in support vector clustering to perform unsupervised machine learning suitable for some of the methods discussed herein.

Regression analysis is a statistical process for estimating the relationships among variables such as proteases and classification accuracy. It includes techniques for modeling and analyzing relationships between multiple variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning. Other suitable ML algorithms include association rule learning, inductive logic programming, and Bayesian networks. Association rule learning may be used for discerning sets of proteases that signify disease state. Algorithms for performing association rule learning include Apriori, Eclat, FP-growth, and AprioriDP. FIN, PrePost, and PPV. Inductive logic programming relies on logic programming to develop a hypothesis based on positive examples, negative examples, and background knowledge. Bayesian networks are probabilistic models that may represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. Whatever machine learning algorithm is used, the classification algorithm may be used to output a heat map, or activity map, that gives an expression level of each enzyme at stages of the disease.

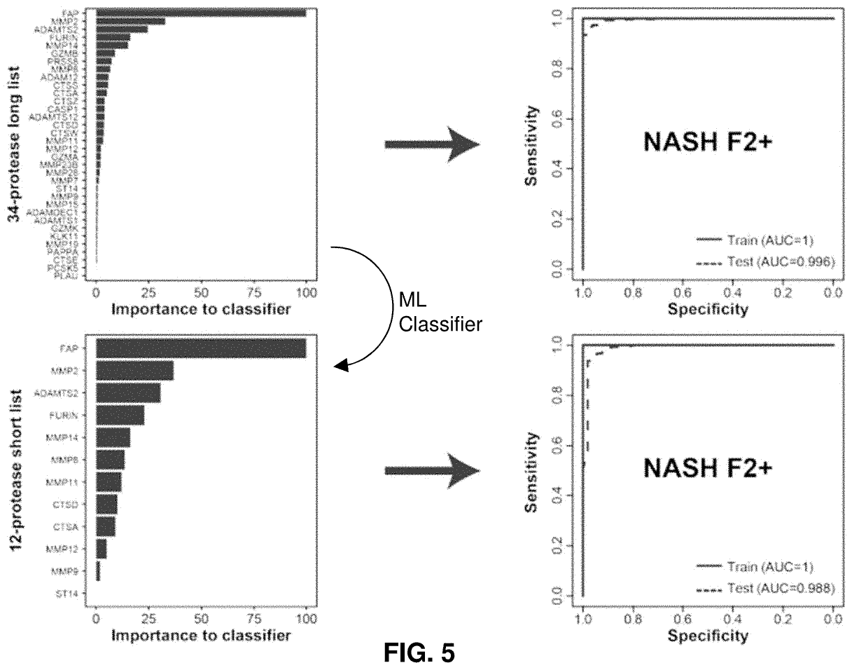

FIG. 5 shows the results of the classification algorithm. The classification algorithm outputs a set of proteases predicted to classify the disease condition with sensitivity and specificity both greater than 0.9. a receiver operating characteristic curve, i.e. ROC curve, is a graphical plot that illustrates the diagnostic ability of a binary classifier system as its discrimination threshold is varied. The accuracy of the test depends on how well the test separates the group being tested into those with and without the disease in question. Accuracy is measured by the area under the ROC curve. An area of 1 represents a perfect test; an area of 0.5 represents a worthless test. A rough guide for classifying the accuracy of a diagnostic test is the traditional academic point system: 0.90-1=excellent (A); 0.80-0.90=good (B); and 0.70-0.80=fair (C). The number is a measure of a test's ability to discriminate correctly and it is computed via methods such as a non-parametric method based on constructing polygons under the curve as an approximation of area or parametric methods using a maximum likelihood estimator to fit a smooth curve to the data points. Both methods are available as computer programs. see Metz C E. Basic principles of ROC analysis. Sem Nuc Med. 1978; 8:283-298. The area measures discrimination, that is, the ability of the test to correctly classify those with and without the disease. Across the top in the depicted figures, the sensitivity is shown to be 0.996 AUC using 34 proteases. The classification algorithm selects the 12 proteases shown in the bottom, giving a sensitivity of 0.988. In both cases, the “training” score is 1, representing the RNA-Seq data that was used as ground truth. A computer system of the disclosure may be used to output the selected set of proteases. In preferred embodiments, the computer system selects the cleavage targets as substrates for the proteases output by the classification algorithm. That is, the output of the computer system may be a list of amino acid sequences, each of which is one cleavage substrate for one of the proteases. That list of amino acid sequences may be presented to a user or used as an input for creating polypeptides.

In the illustrated example, the disease is nonalcoholic steatohepatitis (NASH) and the enzymes include FAP, MMP2, ADAMTS2, FURIN, MMP14, GZMB, PRSS8, MMP8, ADAM12, CTSS, CTSA, CTSZ, CASP1, ADAMTS12, CTSD, CTSW, MMP11, MMP12, GZMA, MMP23B, MMP7, ST14, MMP9, MMP15, ADAMDEC1, ADAMTS1, GZMK, KLK11, MMP19, PAPPA, CTSE, PCSK5, and PLAU. The classification algorithm identified a subset of enzymes (FAP, MMP2, ADAMTS2, FURIN, MMP14, MMP8, MMP11, CTSD, CTSA, MMP12, and MMP9) that uniquely and reliably signify presence of NASH and stage 2 fibrosis (AUC>0.90).

The polypeptides may be formed for inclusion in an activity sensor. Embodiments of the disclosure include providing the polypeptides for assembly in a nanosensor. The polypeptides may be synthesized using, e.g., a reactor instrument for solid phase synthesis. The polypeptides may be ordered from a commercial provider such as Thermo-Fisher Scientific (Waltham, Mass.) or Sigma-Aldrich Corp. (St. Louis, Mo.). These polypeptides will provide the cleavable reporters for activity sensors. Preferably, each cleavable reporter/polypeptide includes a cleavage site for a protease and a detectable analyte that is released from the activity sensor upon cleavage. It may be preferable to include a free sulfhydryl group, e.g., proximal to the cleavage site with the detectable analyte distal to the cleavage site, as a free sulfhydryl group may facilitate covalent linkage to a scaffold of the activity sensor.

Methods of the disclosure further may include creating an activity sensor comprising cleavable reporters that are released as analytes in vivo upon exposure to the subset of enzymes.

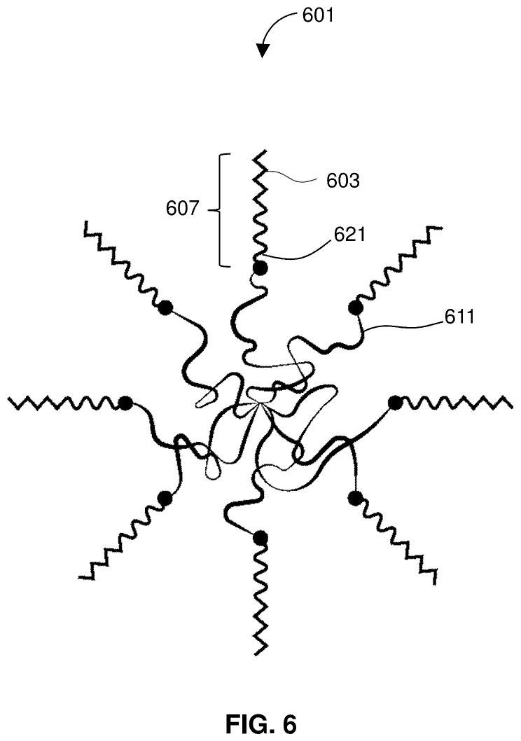

FIG. 6 shows an activity sensor 601. The activity sensor 601 includes a plurality of cleavable reporters 607. Each cleavable reporter 607 includes a cleavage site 621 for a protease and a detectable analyte 603 that is released from the activity sensor 601 upon cleavage. The cleavable reporters 603 are conjugated to polymer scaffold 611. Any suitable polymer scaffold may be used including, for example, scaffolds of biocompatible polymers such as polylactic glycolic acid, collagen, chitin or chitosan, polyethylene glycol (PEG), nucleic acids, sugars, amino acids, or others. For example, the polymer scaffold 611 may include peptidoglycan. In preferred embodiments, the scaffold 611 comprises a plurality of maleimide-PEG subunits. In certain embodiments, the scaffold 611 is a 40 kDa 8-arm PEG scaffold.

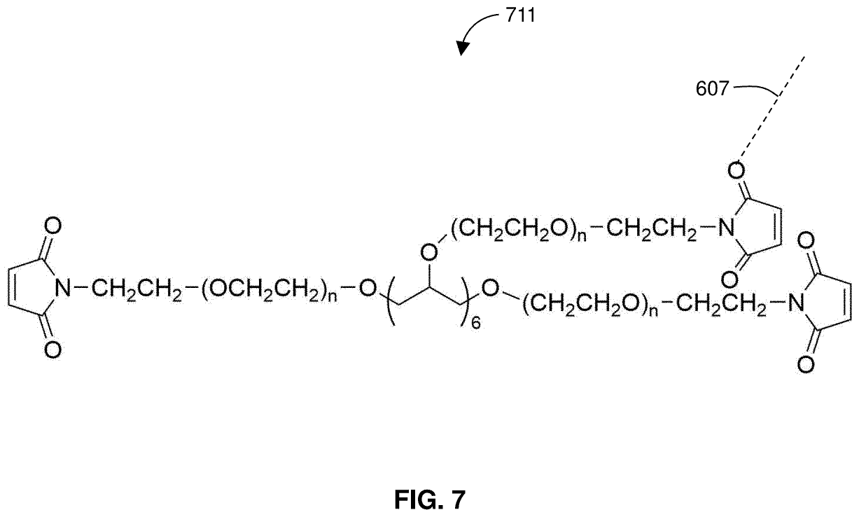

FIG. 7 shows an 8-arm PEG scaffold 711 for use as the scaffold 611, where n is chosen for the mass closest to 40 kDa. A cleavable reporter 607 is preferably linked to the scaffold 711. A size of about 40 kDa was discovered here for the scaffold to give an activity sensor that is retained in tissue long enough for enzymatic activity, but small enough to ultimately move to the tissue and be safe. The disclosure includes the insight that the activity sensors work well when the scaffold 611 is about 40 kDa. For creation of the activity sensor 601, such a PEG scaffold may be obtained from Advanced BioChemicals, LLC (Lawrenceville, Ga.) or Thermo-Fisher Scientific (Waltham, Mass.). The cleavable reporters 607 may be simply and covalently linked to the scaffold 711 using simple mixing and control of pH and temperature according to the instructions from the supplier. Such methods will produce an activity sensor 601 with a plurality of cleavable reporters 607 that each have a cleavage site 621 for a protease and a detectable analyte 603. In certain embodiments (e.g., useful for liver disease such as NASH), the detectable analytes 603 are each uniquely detectable by virtue of a unique mass designed by a selected amino acid sequence unique to each analyte 603.

One of skill in the art would know what peptide segments to include as protease cleavage sites in an activity sensor of the disclosure. One can use an online tool or publication to identify cleave sites. For example, cleavage sites are predicted in the online database PROSPER, described in Song, 2012, PROSPER: An integrated feature-based tool for predicting protease substrate cleavage sites, PLoSOne 7(11):e50300, incorporated by reference. Any of the compositions, structures, methods or activity sensors discussed herein may include, for example, any suitable cleavage site such as the sequences in a database such as PROSPER as cleavage sites, as well as any further arbitrary polypeptide segment to obtain any desired molecular weight. To prevent off-target cleavage, one or any number of amino acids outside of the cleavage site may be in a mixture of the D and/or the L form in any quantity.

In such embodiments, to stage liver disease, the activity sensors 601 can be administered to a patient. For example, the activity sensor can be injected intravascularly. When the activity sensors 601 are administered to the patient in such embodiments, they accumulate in the liver due to their mass. In the liver, the set of proteases cleave the activity sensor 601 at the cleavage sites 621 to thereby release the analyte 603 into the bloodstream. In circulation, the analytes 603 are filtered by the kidneys and excreted in the patient's urine. A sample of the urine may be collected and analyzed for the presence of the detectable analytes.

Where the analytes each have a unique mass by virtue of the design of the polypeptide sequence, mass spectrometry may be performed on the urine sample to reveal the presence or absence of mass spectra signifying the presence or absence of the disease condition in the patient's liver.

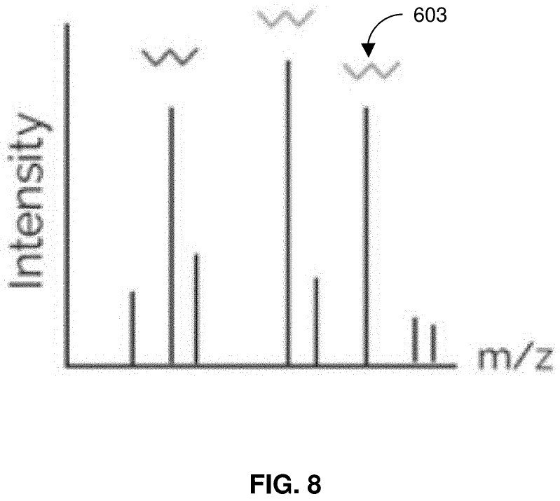

FIG. 8 illustrates an exemplary mass spectra obtained from a patient. The sawtooth lines represent the detectable analytes 603, and that each has a distinguishing mass to charge (m/z) ratio. The presence of the indicated peaks on the mass spectra indicates that the proteases were present in the liver and cleaved the reporters.

Methods of the disclosure provide an analytical pipeline for mapping activity in a disease-specific manner. Any of a variety of diseases or medical conditions may be mapped using the analytical pipeline. In preferred embodiments, the pipeline uses expression data (e.g., from RNA-Seq) to identify proteases that are active in disease tissue and subject to differential expression relative to normal tissue. A machine learning classifier selects a subset of the proteases that identify the disease with a threshold sensitivity and specificity, in which the subset is small enough that a corresponding set of protease substrates may be assembled into a nanoparticle activity sensor that, when administered to a patient, are cleaved in the disease tissue to release detectable analytes signifying presence of the disease. Any suitable disease may be activity-mapped according to the methods including, for example, cancer; osteoarthritis; and infection by a pathogen.

Methodologies herein and the informatics pipeline may be provided by a computer system that performs steps of the methods.

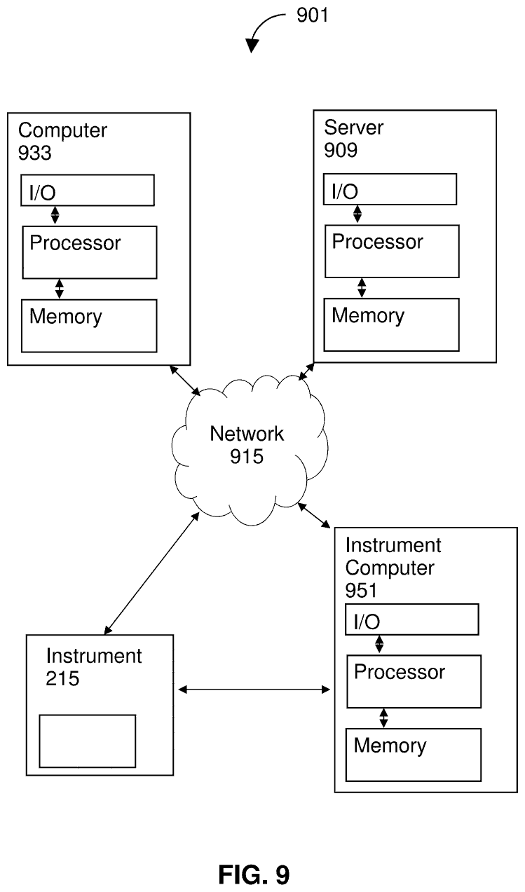

FIG. 9 illustrates a system 901 according to certain embodiments. The system 901 includes at least one computer 909 comprising a processor coupled to memory having instructions therein executable by the processor to cause the system to analyze gene expression of tissue in a disease state to identify enzymes differentially expressed in the tissue compared to healthy tissue and select a subset of the enzymes that correlates with the disease state to threshold sensitivity or specificity. The system stores or outputs a set of enzymes specific for a disease or even for a stage of a disease, or specific design parameters for the activity sensor, such as polypeptide sequences to be included for cleavage by the enzymes. The system may include instruments such as nucleic acid sequencing instrument 215 to perform RNA-Seq to determine the gene expression levels from the tissue. wherein analyzing the gene expression includes sequencing RNA from disease tissue samples to produce transcript sequences. The system may use the transcript sequences, or translations thereof, to query a gene or protein database to identify candidate proteases. The system may include a user computer 933 by which a user initiates the processes or procures results. A sequencing instrument 215 may itself have (e.g., onboard) a computer 951 that plays a role in analyzing or assembling sequences. While discussed herein generally in terms of activity sensors that are themselves substrates for protease activity in disease tissue, other embodiments may be within the scope of the disclosure.

For example, in some embodiments, the informatics pipeline of the disclosure is used in the design of nanosensors that employ nucleases that exhibit catalytic cleavage to report the presence of certain sets of nucleic acid sequences in tissue.

Collateral cleavage-based embodiments of the disclosure provide methods for designing activity sensors that include analyzing gene expression data for tissue affected by a disease condition to identify candidate genes differentially expressed in the tissue compared to healthy tissue; identifying a set of signature genes that classify the disease condition with a threshold sensitivity or specificity; and creating a composition that, when administered to the subject, releases one or more detectable reporters in the presence of nucleic acid sequences of the signature genes. The composition may include a Cas protein such as Cas13 that exhibits collateral cleavage in the presence of the nucleic acid sequences of the signature genes. Preferably, the composition includes reporters that include quenched fluorophores that fluoresce in response to collateral cleavage by the Cas protein. Optionally, the composition includes a plurality of the Cas proteins, and the composition provides a fluorescent signature that classifies the disease based on exposure of the Cas proteins to the nucleic acid sequences of the signature genes.

### EXAMPLES

### Example 1: Protease Expression in Patients with NASH

Hepatic protease expression in patients with NASH correlates with fibrosis stage and treatment response.

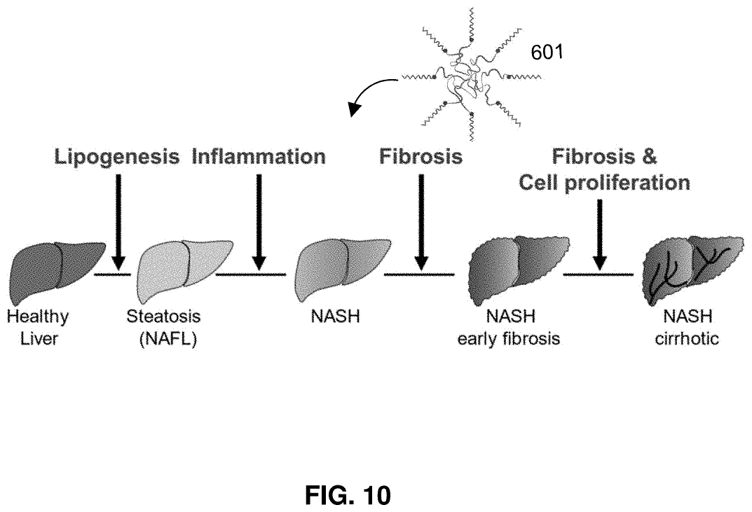

FIG. 10 shows progression through fibrosis and a stage at which an activity sensor 601 created by the method 101 may be used to detect disease. When designed according to the informatics pipeline, the activity sensor will report any specific stage of the disease. Proteases involved in fibrosis, inflammation, and cell death may be important in the progression of NASH. A pipeline method 101 may be used in developing protease nanosensors 601 designed to assess NASH disease severity and monitor treatment response.

RNA sequencing (RNA-Seq) is performed on RNA extracted from procured formalin fixed and paraffin embedded (FFPE) liver tissue from patients with NASH (all NAS≥3) and hepatic fibrosis as well as healthy controls. Additionally, RNA-Seq is performed on RNA extracted from fresh liver tissue obtained at baseline (BL) and weeks later (W) from subjects with NASH (all NAS≥5) and F2 or F3 fibrosis treated with one or more therapeutics. Protease gene expression is compared between NASH patients and controls. Associations between protease gene expression and fibrosis stage, as well as changes in gene expression according to fibrosis response (≥1-stage improvement) between BL and W, are evaluated.

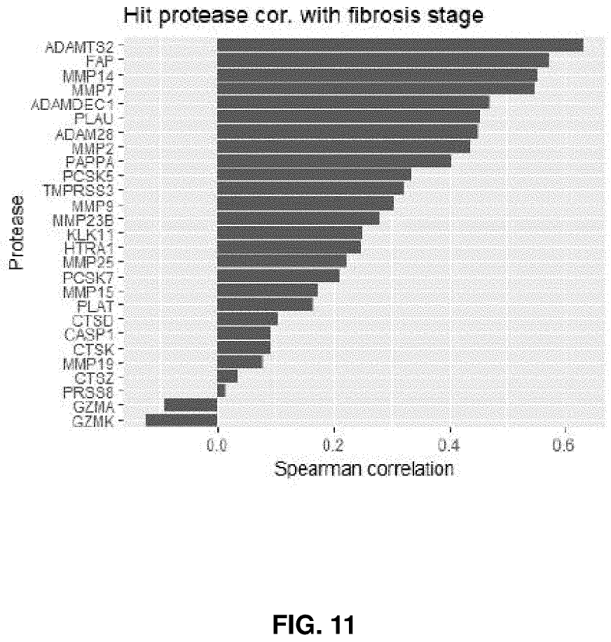

FIG. 11 shows protease “hits”, proteases that exhibit differential expression that correlates with fibrosis stage.

NASH-integral proteases from multiple disease pathways including fibrosis, inflammation, and cell death are identified. The expression levels of 9 protease genes, including FAP, ADAMTS2, MMP14, and MMP15, are increased in NASH patients versus healthy controls (all P<0.05). Additionally, the expression levels of 18 protease genes is positively correlated with fibrosis stage (P<0.05). Between BL and W, the expression of 7 proteases decreased (P<0.05) in patients with fibrosis response compared with non-responders. Compared to all genes, decreases in target proteases were enriched in fibrosis responders vs non-responders (P=0.0014).

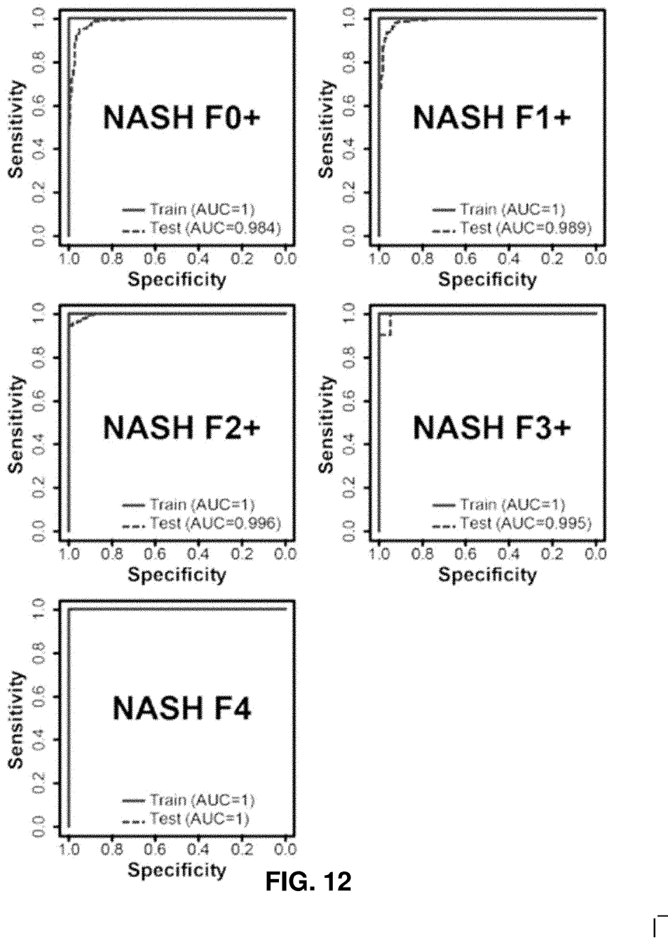

FIG. 12 shows the results of staging NASH using activity sensors for stages F0, F1, F2, F3, and F4. Hepatic protease expression correlates with fibrosis stage and anti-fibrotic response to treatment in patients with NASH.

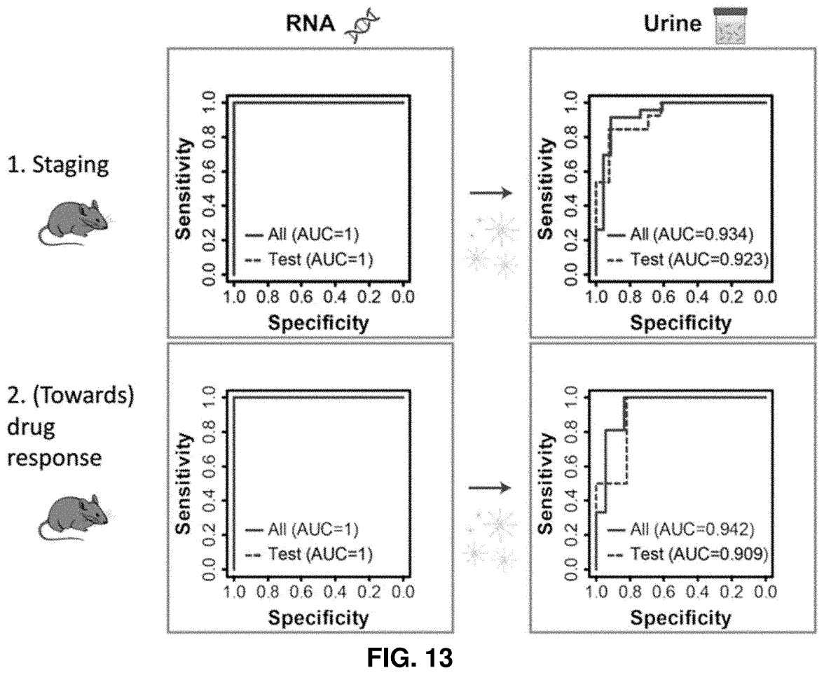

FIG. 13 shows results from validating the activity sensors in mice. Testing shows that activity sensors can stage disease in mice or predict drug response. In each case, the AUC from RNA is 1 because that is taken as the ground truth, and an informatics pipeline and method 101 may be used to design the proteases sensors that detect a result in urine. In mouse tests, the diseases may be staged via the activity sensors to a specificity of 0.934 AUC. For drug response, AUC is 0.942. The method 101 may be useful for designing high sensitive activity sensors. Thus methods 101 and the informatics pipeline may be used to design protease nanosensors that measure the kinetics of proteolysis for the staging and monitoring of treatment response in patients with a disease.

### Example 2: Lung Cancer

Methods are performed to identify candidate proteases upregulated in human cancer. A dataset such as mRNA sequencing (RNA-Seq) and clinical data collected from lung cancer patients may be analyzed using a list of 168 candidate human extracellular proteases generated by UniProt, to determine gene expression levels in the patients.

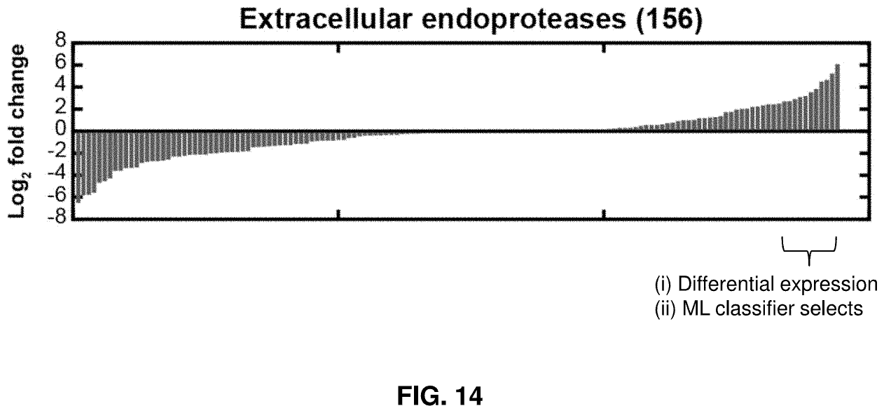

FIG. 14 shows the 156 differentially expressed extracellular proteases for which RNA-Seq data from lung cancer and matched normal adjacent tissue are available. The 156 proteases in the dataset with RSEM (RNA-Seq by Expectation Maximization) have counts sufficiently high to perform differential expression analysis, with log 2 fold change expressions distributed between approximately −7 and +6 in tissue classified as LUAD compared to normal adjacent tissue. An informatics pipeline preferably uses the RNA-Seq data to retrieve identities of those proteases. A machine learning classifier in the pipeline preferably converges on a small set of signature proteases.

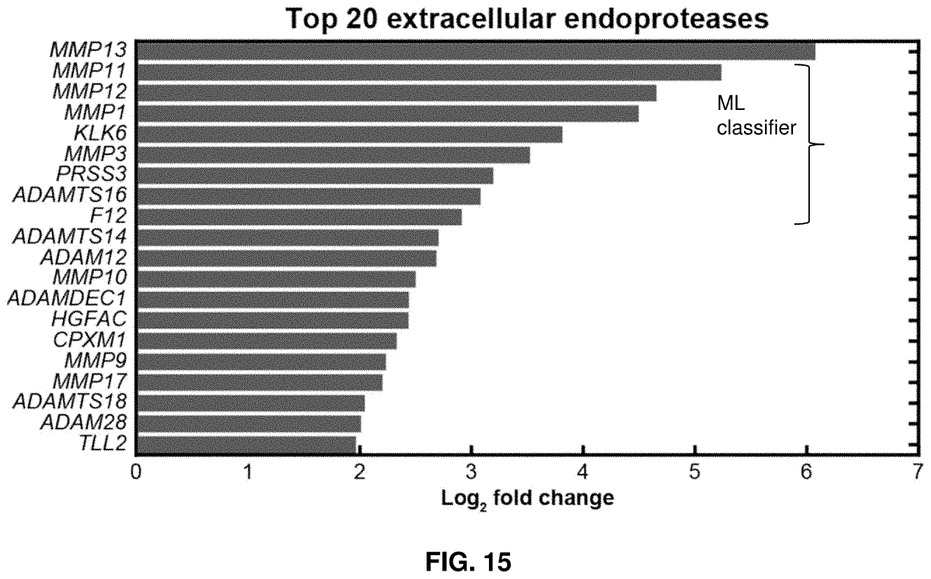

FIG. 15 shows the 20 most highly upregulated genes revealed eight members of the matrix metalloproteinase (MMP) family and five members of the disintegrin and metalloproteinase (ADAM) family. Differential expression of key proteases are a potential means to assess disease stage. In the depicted embodiment, the disease is lung cancer, and the subset of enzymes includes MMP13, MMP11, MMP12, MMP1, KLK6, MMP3 and others.

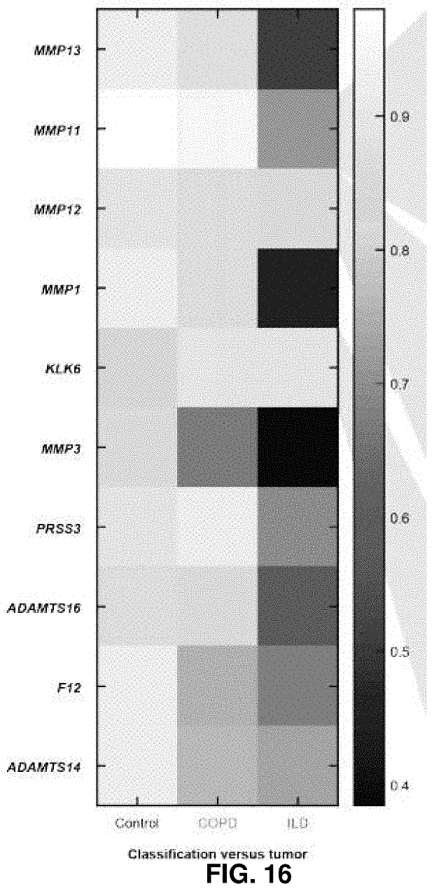

FIG. 16 shows an activity map, or heat map, generated from analysis of RNA-Seq data from related pathologies [chronic obstructive pulmonary disease (COPD) and interstitial lung disease (ILD). The heat map shows that protease signatures may be useful for differentiation between LUAD and benign pulmonary pathologies.

Methods of the disclosure are tested in a relevant mouse model, a genetically driven model of adenocarcinoma (a type of NSCLC that accounts for 37.8% of all cases of lung cancer) (SEER Cancer Statistics Review, 1975-2011, 2014) that incorporates mutation in those genes. The model uses intra-tracheal administration of adenovirus expressing Cre recombinase (adeno-Cre) to activate mutant KrasG12D and delete both copies of p53 in the lungs of KrasLSLG12D/+;Trp53fl/fl (KP) mice, initiating tumors that closely recapitulate human disease progression from alveolar adenomatous hyperplasia to grade IV adenocarcinoma over the course of weeks. The proteolytic landscape of the KP model is characterized to assess homology to that of human lung cancer. Transcriptomic data for the KP model is analyzed to identify overexpressed, secreted proteases.

Both metastatic (n=9) and non-metastatic (n=10) primary tumor samples are pooled and compared to normal lung (n=2). While some of the top 10 overexpressed proteases in human lung cancer are also found to be overexpressed in the KP model, others are not. Furthermore, some proteases demonstrated stage-specific upregulation. An inhaler-based mechanism is developed to deliver protease sensitive nanoparticles (the activity reporters) directly to the lung. Pulmonary drug delivery is typically accomplished by inhalation of aerosols (usually by metered dose inhaler or nebulizer) or dry powders (usually by dry powder inhaler). A pressure-driven aerosolization device may be used for its ease of use, deep lung penetration, and delivery capacity. With this technique, activity sensors are directly aerosolized and transmission electron microscopy (TEM) on 40 kDa eight-arm poly(ethylene glycol) (PEG-8 [40 kDa]) carrier particles before and after aerosolization revealed no aggregation or other changes in appearance.

Analysis of proteolytic cleavage of a FRET-paired, MMP-sensitive nanosensor by enzymes MMP2 and MMP13 in vitro demonstrates no difference in fluorogenic cleavage between particles pre- and post-aerosolization, suggesting that aerosolized nanoparticles retain both their size and functionality following lung deposition by aerosolization.

The method 101 and the informatics pipeline is preferably used to design fourteen nanosensor variants that use a panel of MMP-sensitive peptide substrates that release mass-encoded reporters upon proteolysis. For each variant, the ML classifier may provide the panel of substrates. The activity sensors are created and include protease-sensitive peptide substrates bound to PEG-8 [40 kDa]. Following substrate proteolysis, the small reporters cross into the bloodstream, where they are concentrated into the urine by glomerular filtration. Reporters are designed to yield uniquely detectable peaks by mass spectrometry.

The PEG-8 [40 kDa] nanosensor scaffold (designed to be retained in the lung) and the small free urinary reporter (designed to filter efficiently into the urine) upon introduction by inhalation or intravenous injection is compared. Using ELISA compatible PEG-8 40 kDa scaffold and free reporter, urine is collected up to 60 minutes post-dose and quantified by ELISA. As expected due to its large size compared to glomerular porosity (˜10 nm particle size vs ˜5 nm glomerular filtration limit), urinary scaffold concentrations were ˜5,000-fold lower than the injected and inhaled dose (50.0 pM by aerosol and 51.4 pM by intravenous injection; P=1.00). In contrast, the small 2.4 kDa free reporter was substantially present in the urine within 60 minutes post-dose by both pulmonary and intravenous delivery (157.9 nM by aerosol and 513 nM by intravenous injection; P=0.007), indicating the reporters are rapidly and efficiently partitioned from the lung into the blood and subsequently from the blood into the urine.

Multiplexed, protease-sensitive activity sensors are administered to KP mice and control mice 7.5 weeks after tumor initiation, when lung tumors are 1-2 mm in diameter. For those experiments, activity sensors are administered by intra-tracheal intubation. Urine is collected one hour after inhalation and liquid chromatography followed by tandem mass spectrometry (LC-MS/MS) is performed. Reporters may be normalized to account for any differences in inhalation efficiency or urine concentration. Using this system, a three-reporter classifier provides accurate discrimination of disease mice from control mice at 7.5 weeks, an unexpected finding given the insensitivity of the gold standard detection tool, microCT, at this time point. See Haines, 2009, A quantitative volumetric micro-computed tomography method to analyze lung tumors in genetically engineered mouse models, Neoplasia 11(1):39-47, incorporated by reference. The data demonstrate the power of multiplexed, inhalable protease activity sensors in detecting lung cancer at the earliest stages of tumor development.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for designing an activity sensor, the method comprising:
analyzing gene expression of tissue in a disease state to identify enzymes expressed in the tissue under a specific physiological state or health condition;
selecting a subset of the enzymes that correlates with the physiological state or health condition to a predefined performance metric; and
creating an activity sensor comprising cleavable reporters that are released as analytes in vivo upon exposure to the subset of enzymes.

2. The method of claim 1, wherein the enzymes are proteases.

3. The method of claim 2, wherein when the activity sensor is administered to a subject, proteases cleave the activity sensor in the tissue under the physiological state to thereby release the analyte for collection in a bodily sample.

4. The method of claim 1, wherein the subset of enzymes is selected by a machine learning classification algorithm that classifies subsets by whether they meet the performance metric, wherein the performance metric includes a defined threshold sensitivity or specificity.

5. The method of claim 4, wherein the physiological state is a disease, and the classification algorithm outputs a heat map that gives an expression level of each enzyme for each of a plurality of substrates and/or stages of a disease condition.

6. The method of claim 5, wherein the classification algorithm outputs a set of proteases predicted to classify the disease condition with sensitivity and specificity both greater than 0.90.

7. The method of claim 6, further comprising selecting the cleavage targets as substrates for the proteases output by the classification algorithm.

8. The method of claim 1, wherein analyzing the gene expression includes sequencing RNA from disease tissue samples to produce transcript sequences.

9. The method of claim 8, further comprising comparing the transcript sequences, or translations thereof, to a gene or protein database to identify candidate proteases.

10. The method of claim 8, wherein the samples comprise formalin-fixed slices from tumors.

11. The method of claim 1, wherein the enzymes are proteases and creating the nanoparticles comprises linking a plurality of peptides to a polymer scaffold.

12. The method of claim 11, wherein each of the peptides comprises a detectable analyte linked to the scaffold via a cleavage target of one of the signature proteases.

13. The method of claim 12, wherein the polymer scaffold comprises a multi-arm PEG) structure.

14. The method of claim 1, wherein administering the activity sensor to a subject yields a bodily sample from the subject that includes the analytes, indicating disease activity before other disease symptoms are exhibited by the subject.

15. The method of claim 1, wherein the disease is nonalcoholic steatohepatitis (NASH), the enzymes comprise FAP, MMP2, ADAMTS2, FURIN, MMP14, GZMB, PRSS8, MMP8, ADAM12, CTSS, CTSA, CTSZ, CASP1, ADAMTS12, CTSD, CTSW, MMP11, MMP12, GZMA, MMP23B, MMP7, ST14, MMP9, MMP15, ADAMDEC1, ADAMTS1, GZMK, KLK11, MMP19, PAPPA, CTSE, PCSK5, and PLAU, and the subset of enzymes comprises a plurality of FAP, MMP2, ADAMTS2, FURIN, MMP14, MMP8, MMP11, CTSD, CTSA, MMP12, and MMP9.

16. The method of claim 1, wherein the disease comprises lung cancer, and the subset of enzymes includes MMP13, MMP11, MMP12, MMP1, KLK6, and MMP3.

17. The method of claim 1, wherein the disease comprises one selected from the group consisting of a cancer; osteoarthritis; and infection by a pathogen.

18. The method of claim 1, wherein the enzymes are proteases and the method includes determining subsets of the proteases specific to disease stages, wherein administering the activity sensor to a subject yields a bodily sample with analytes indicative of a stage of the disease.

19. A method for designing activity sensors, the method comprising:
analyzing gene expression data characteristic of a disease condition to identify candidate genes differentially expressed under the disease condition;
identifying a set of signature genes that classify the disease condition; and
creating a composition that, when administered to the subject, releases one or more detectable reporters in the presence of nucleic acid sequences of the signature genes.

20. The method of claim 19, wherein the composition includes a Cas protein that exhibits collateral cleavage in the presence of the nucleic acid sequences of the signature genes.

21. The method of claim 20, wherein the composition includes reporters that include quenched fluorophores that fluoresce in response to collateral cleavage by the Cas protein.

22. The method of claim 20, wherein the composition includes a plurality of the Cas proteins, and the composition provides a fluorescent signature that classifies the disease based on exposure of the Cas proteins to the nucleic acid sequences of the signature genes.
